(19) United States
(12) Patent Application Publication
(10) Pub. No.: US 2019/0008913 A1
(43) Pub. Date: Jan. 10, 2019

(54) Topical Treatment Compositions Comprising Benzoin and Olive Fruit Extract

(71) Applicant: Elizabeth Laura DENTE, New York, NY (US)

(72) Inventor: Elizabeth Laura DENTE, Englewood, NJ (US)

(21) Appl. No.: 16/007,454

(22) Filed: Jun. 13, 2018

Related U.S. Application Data
(63) Continuation of application No. 14/560,776, filed on Dec. 4, 2014, now abandoned.
(60) Provisional application No. 61/918,333, filed on Dec. 19, 2013.

(51) Int. Cl. A61K 36/63; A61Q 19/08; A61K 8/35; A61K 8/34; A61Q 19/02; A61Q 19/00; A61Q 17/00; A61K 31/121; A61K 31/12; A61K 31/05; A61K 8/92; A61Q 19/10

(52) U.S. Cl. CPC A61K 36/63; A61Q 19/10; A61K 8/35; A61K 8/347; A61Q 19/02; A61Q 19/008; A61Q 19/007; A61Q 19/004; A61Q 19/00; A61Q 17/005; A61K 31/121; A61K 31/12; A61K 31/05; A61K 8/922; A61K 2800/522; A61Q 19/08

(57) ABSTRACT

Disclosed are treatment compositions adapted for application to mammalian skin, which treatment compositions are effective initiators for the generation of human keratinocytes on or within the treated skin, particularly in the locus of the skin wherein the treatment composition has been applied, which treatment composition comprises benzoin and an olive oil extract which contains hydroxytyrosol and/or hydroxytyrosol. In certain preferred embodiments the benzoin and the olive oil extract and/or hydroxytyrosol are microencapsulated.

The present invention relates to compositions for topical application to mammalian skin, and to topical treatment methods. More particularly the present invention relates to topically applied compositions which are useful in the general treatment of mammalian (human, animal) skin to induce the generation of human keratinocytes.

Skin acts as the mammalian body's shield and is continually being remodeled in order to retain maximum protection. The extracellular matrix (ECM) of skin is the network between skin cells that maintains this shield. The ECM provides support and anchorage for skin cells, giving strength and elasticity to the skin. Under normal conditions, the enzyme family matrix metalloproteinases (MMPs) are present in skin to facilitate natural remodeling to the ECM. When the skin suffers any wound, our body's reaction is to overproduce (MMPs) and inflammatory cytokines. Indeed MMPs are most directly affected by environmental stress and trauma. This overproduction further stimulates oxidative stress and the presence of free radicals in cells. This reaction by the body to a wound is described as out-of-control and destructive to the ECM. MMP-9, specifically, is a key agent of the degradation done to the skin following trauma. Fibronectin, a glycoprotein present in the extracellular matrix, plays a major role in wound healing and cell adhesion. Fibronectin is active mostly in the epidermis layer of the skin. Collagen and elastin, both found most commonly in the dermis, are proteins that are also involved in wound healing. These proteins provide firmness and strength to the skin and its extracellular matrix. In the presence of a wound, these three proteins are targeted and broken down by the increased levels of MMPs and free radicals. The increased levels of MMPs and free radicals increase the degradation to the extracellular matrix, while also decreasing the amount of repair done by diminishing levels of collagen, fibronectin, and elastin.

Trauma to the skin is worsened by oxidative stress, in which free radicals cause great damage in stressed cells. Currently, natural antioxidants are highly sought after for wound repair treatments for their free radical scavenging abilities. Olive fruit extract contains the powerful antioxidant, hydroxytyrosol.

Notwithstanding the foregoing, the present inventor has found that compositions, e.g, topical compositions, which contain both benzoin and olive fruit extract are synergistically and/or surprisingly effective in inducing the generation of human keratinocytes.

In a first aspect the present invention provides treatment compositions adapted for application to mammalian skin, which treatment compositions are effective initiators for the generation of human keratinocytes on or within the treated skin, particularly in the locus of the skin wherein the treatment composition has been applied.

In a second aspect the present invention provides a topical treatment composition, which topical treatment composition is effective in inducing the generation of human keratinocytes on the treated skin, particularly in the locus of the skin wherein the treatment composition has been applied.

According to a third aspect of the invention the invention provides topical treatment compositions adapted for application to mammalian skin, which treatment compositions are effective initiators for the generation of human keratinocytes on or within the treated skin, particularly in the locus of the skin wherein the treatment composition has been applied, which treatment compositions comprise both benzoin and hydroxytyrosol, (which hydroxytyrosol may be provided as an olive oil extract, or which may be refined therefrom or which hydroxytyrosol may be synthetically produced).

A fourth aspect of the present invention is a skin repair composition according to the aforesaid first, second or third inventive aspects.

A fifth aspect of the invention is a wound treatment composition for the treatment of topical wounds and scars according to the first through fourth aforesaid aspects of the invention.

In a sixth aspect of the invention there is provided an encapsulated composition which composition comprises both encapsulated, preferably microencapsulated benzoin and microencapsulated hydroxytyrosol, (which hydroxytyrosol may be provided as an olive oil extract, or which may be refined therefrom or which hydroxytyrosol may be synthetically produced), microencapsulated benzoin and microencapsulated olive oil extract which latter material contains hydroxytyrosol.

A seventh aspect of the invention is a composition according to any of the first through fourth aspects, which comprises an encapsulated composition which comprises both benzoin and olive oil extract which latter material contains hydroxytyrosol.

In an eighth aspect of the invention there is provided a composition according to any of the first through fourth aspects, which comprises an encapsulated composition which includes both benzoin and hydroxytyrosol, which hydroxytyrosol may be provided as an olive oil extract, or which may be refined therefrom or which hydroxytyrosol may be synthetically produced.

A ninth aspect of the invention is a method for the treatment of the mammalian skin, which method comprises the step of: applying a composition according to any of the aforesaid aspects of the invention to mammalian skin, e.g., human skin. Further preferably the process comprises the further step of inducing, increasing or initiating the generation of human keratinocytes on or within the treated skin, particularly in the locus of the skin wherein the treatment composition has been applied.

A still further aspect of the invention is a personal care product according to any of the first through sixth aspects of the invention described above.

In a further aspect of the invention there is provided a topical skin treatment product, and/or a wound treatment product comprising a composition according to any of the first through sixth aspects of the invention described above.

These and further aspects and features of the present invention will become more apparent from reading the following description.

FIG. A depicts a graph of the retention of an encapsulated active material.

FIG. B illustrates a depiction of various surface packing effects of capsules.

FIG. C illustrates a scanning electron microscope (SEM) of capsules.

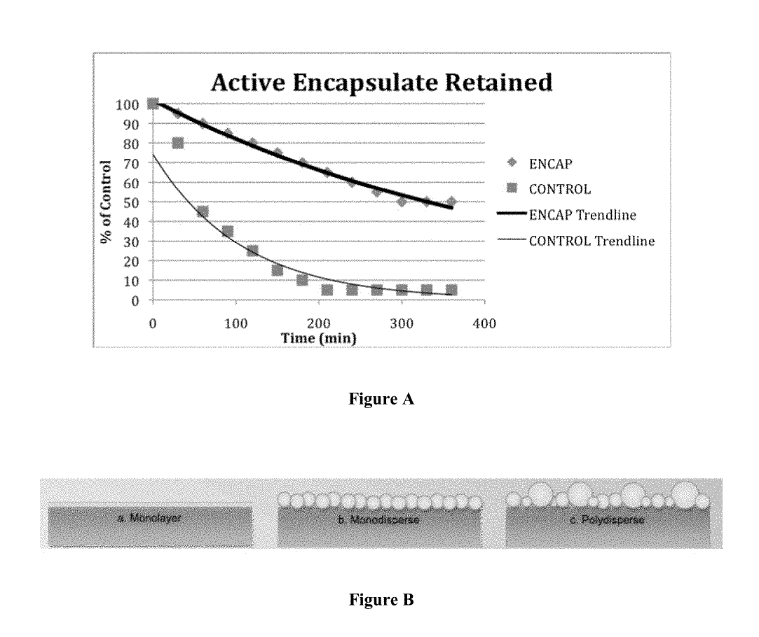

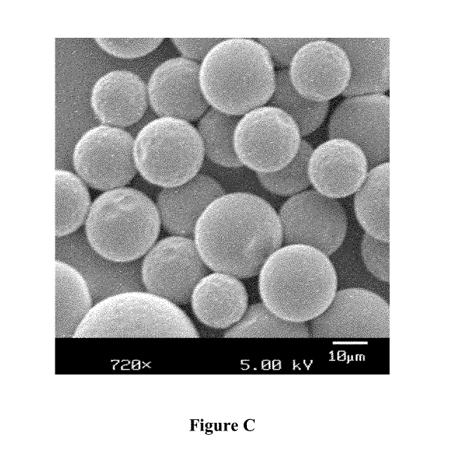

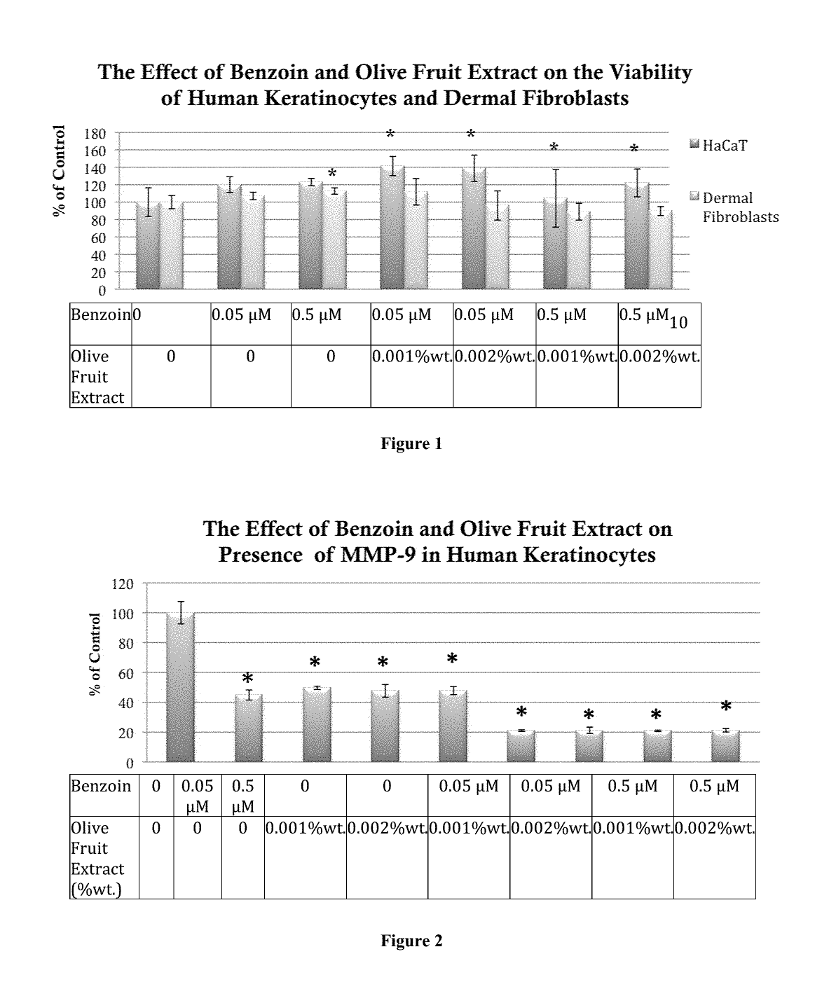

FIG. 1 is a graphical representation of the effect of bezoin and olive fruit extract on the viability of human keratinocytes and dermal fibroplats.

FIG. 2 is a graphical representation of the effect of benzoin and olive fruit extract on the presence of MMP-9 in human keratinocytes.

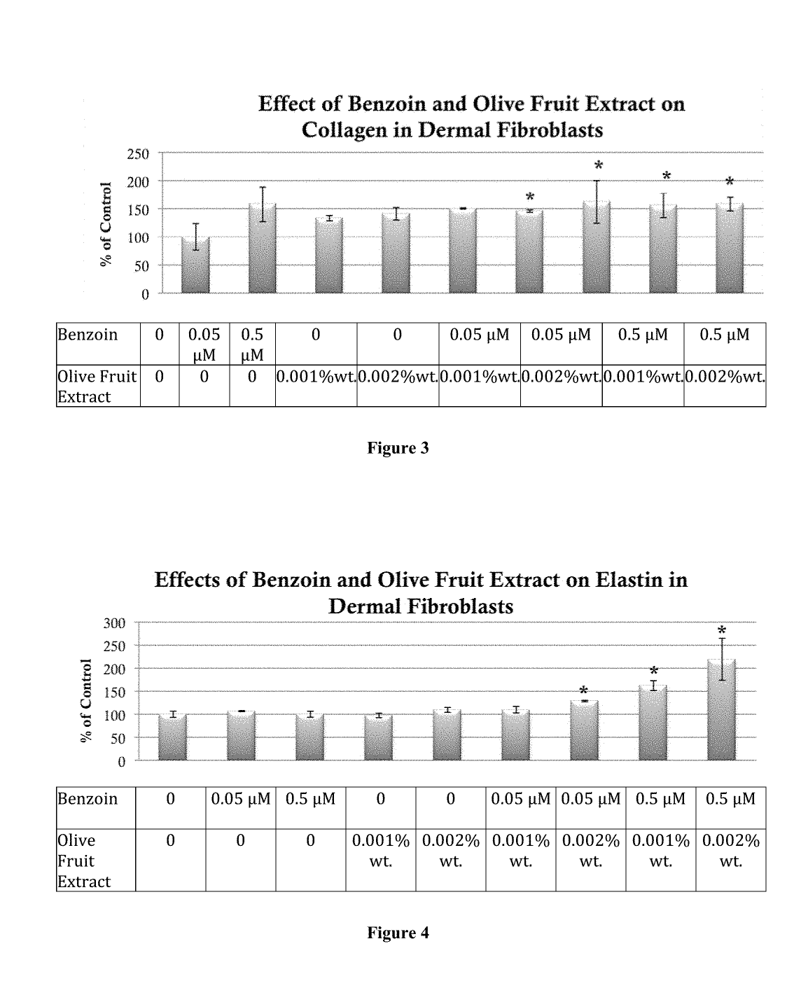

FIG. 3 is a graphical representation of the effects of benzoin and olive fruit extract on collagen in dermal fibroplasts.

FIG. 4 is a graphical representation of the effects of benzoin and olive fruit extract in dermal fibroplasts.

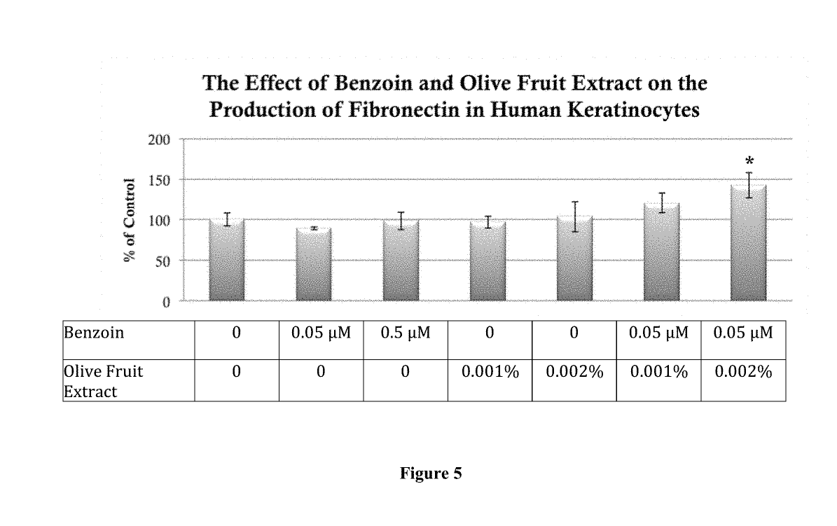

FIG. 5 is a graphical representation of the effects of benzoin and olive fruit extract on the production of fibronectin in human keratinocytes.

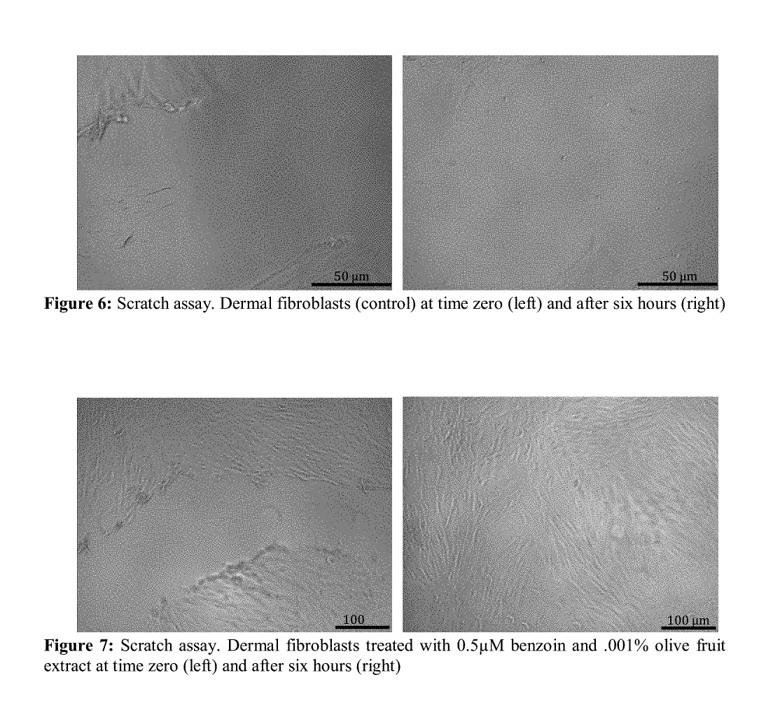

FIG. 6 are photographs of scratch assays (control).

FIG. 7 are photographs of scratch assays of dermal fibroplasts treated with benzoin and olive fruit extract.

The wound repair properties of the organic compound benzoin and the antioxidant olive fruit extract, in combination, have never before evaluated. In addition, the ability of benzoin to have wound healing properties on the skin has, prior to the present invention, not been scientifically demonstrated.

Olive fruit extract contains the antioxidant hydroxytyrosol, which can prevent the damage caused by oxidative stress and free radicals, which are induced in skin by a severe wound. Matrix metalloproteinase-9 (MMP-9), an enzyme in the MMP family, is involved in the degradation that occurs when the skin is wounded and as it ages over time. When the activity of MMP-9 is inhibited, the skin can maintain a healthier structure and recover from wounds more rapidly. In addition, fibronectin is a glycoprotein present in the extracellular matrix that plays a major role in wound healing. Collagen and elastin, present in the dermis, are proteins necessary for wound healing and skin elasticity.

It was believed by the inventor that the combination of benzoin and an olive fruit extract which contains the antioxidant hydroxytyrosol would decrease the levels of MMP-9 in human keratinocytes and increase the levels of fibronectin, collagen, and elastin in dermal fibroblasts in a dose-dependent manner. The hypothesis was tested, using as model cell lines, HaCaT, a human keratinocyte cell line which originates in the epidermis layer of skin, and dermal fibroblasts, which originates in the dermis of skin. A viability assay (using MTS, a tetrazolium compound, and an electron coupling reagent) showed that all concentrations of benzoin and olive fruit extract used were nontoxic. Furthermore, the combination of benzoin and olive fruit extract the antioxidant hydroxytyrosol was surprisingly found to induce a statistically significant (p<0.05) proliferation in the HaCaT cell line. Olive fruit extract which was standardized to contain 25% hydroxytyrosol, reduced 2,2-diphenyl-1-picrylhydrazyl (DPPH) in a dose-dependant manner, exhibited exceptional antioxidant ability at all concentrations. Statistically significant (p<0.05) enzyme-linked immunosorbent assay (ELISA) results showed that MMP-9 levels in HaCaT decreased as the concentrations of benzoin and olive fruit extract increased. The benzoin and the olive fruit extract containing a high proportion of hydroxytyrosol, when combined, demonstrated a significant upregulation of collagen and elastin in dermal fibroblasts as well as a trend towards a modest increase of fibronectin in human keratinocytes. These results demonstrate that the combination of benzoin and the olive fruit extract having a high proportion of hydroxytyrosol, when applied to skin, lessens the degradation done to the skin in a severe trauma. This is caused by the concurrent ability of the benzoin and olive fruit extract combination to downregulate MMP-9 and to upregulate fibronectin, collagen and elastin in skin cells.

Treatment compositions, and topical compositions, using a composition of both benzoin and olive fruit extract containing hydroxytyrosol provide a unique skin (dermal, epidermal) treatment benefit when topically applied, and further, such compositions are particularly effective in providing wound healing effects on skin to which it has been applied, particularly at or near the locus of a wound. Further, good treatment of scars, skin scars and scarred dermal tissue may be provided by the use of said treatment compositions. Alternatively such treatment compositions and topical compositions providing the aforesaid benefits may also be provided wherein both benozin and hydroxytyrosol is present, and the hydxroxytyrosol is separated or extracted from olive oil and/or wherein the hydroxytyrosol is synthetically produced. In either of these foregoing alternatives, preferably the benzoin and hydroxytyrosol are provided at a relative ratio (wt %:wt %) in the range set forth below.

The combination of benzoin and olive fruit extract containing hydroxytyrosol, and/or the combination of benzoin and hydroxytyrosol, is expected to be useful in treatment compositions, i.e., topical treatment compositions. In preferred embodiments the foregoing compositions are used as compositions for skin repair, i.e., wound treatment. While treatment compositions which solely comprise benzoin and olive fruit extract may be used and form an aspect of the invention, optionally but preferably the treatment compositions include one or more of any of a number of known art materials which may be useful in topical compositions and which do not deleteriously affect the advantageous properties the benzoin and olive oil extract and/or the combination of benzoin and hydroxytyrosol. Non-limiting examples of such materials include one or more of: liquid or gel carriers, such as water, organic solvents, inorganic solvents, thickeners; surfactants including known anionic, nonionic, cationic, amphoteric and zwitterionic surfactants; soaps such as anionic soaps; thickeners and thickening agents such as clays, acrylic polymer thickeners, naturally occurring and modified celluloses; humectants; silicone conditioning agents such as linear and cyclic silicones, e.g, polysiloxanes; abrasives, e.g, comminuted polymeric polymers, comminuted organic abrasive materials, inorganic particulate materials; sunscreen agents; ultraviolet light absorbing agents; vitamins; antioxidants; enzymes; fragrances, pH adjusting agents, pH buffering compounds, organic acids, inorganic acids, bases, antimicrobial agents such as phenolic antimicrobial effective compounds, non-phenolic antimicrobial effective compounds; preservatives; colorants, e.g. pigments, dyes; non-abrasive particulate fillers; preservatives; and propellant when the composition is provided as a pressurized or aerosol composition.

The treatment compositions of the invention may be provided in any of a variety of forms and product formats. Non-limiting examples of forms include: liquids, thickened liquids, as well as viscous flowable liquids, e.g., gels, pastes, salves, crémes, etc. Further forms include sprayable compositions which may be foaming or non-foaming sprayable compositions. Such sprayable compositions may be pressurized with an aerosol, or may be non-pressurize but which may be pumped such as with a manually operable trigger spray. Other useful forms include solid tablets, cakes or blocks, such as bar soaps. Non-limiting examples of product formats include: shampoo, conditioner, shower gel, hair spray, hair pomade, hair gel, bodywashes, liquid soaps, or other personal care products which may be topically applied. Further non-limiting examples of product formats include tablets and bars, e.g, bar soap, and pressurized or pumpable, sprayable compositions.

As the treatment compositions of the invention may also be used in the treatment of wounds, in a further aspect there are provided wound dressings, e.g, bandages, pads, and the like which have absorbed or adsorbed thereon a quantity of the treatment compositions disclosed hererin.

In a further aspect, the invention includes compositions to which both benzoin and olive oil extract and/or the combination of benzoin and hydroxytyrosol are added as constituent, or in which both benzoin and olive oil extract and/or the combination of benzoin and hydroxytyrosol, are otherwise included. Preferably such include personal care compositions and/or topical treatment compositions including: face cleanser, anti-acne face or body wash, soap product, exfollient, antifungal, antiseptic, moisturizers, sunscreens, or vitamin supplement to provide soothing, moisturizing, anti-acne, anti-bacterial, anti-aging, exfoliating and antioxidant properties. Cosmetic compositions which include both benzoin and olive oil extract are also considered to fall within the scope of the present invention, and such need not necessarily be topical compositions. Non-limiting examples of such cosmetic compositions include lipstick, lip balms, cosmetic pencils, eyeliners, mascara, eyelash treatment products, as well as other cosmetic products useful in the treatment of the face, hands, or skin elsewhere on the human body. Cosmetic products also include lip-plumping product, or any other cosmetic product which may be useful in providing collagen-boosting properties.

In a further aspect of the invention there is provided an encapsulated composition which comprises both benzoin and olive oil extract which latter material contains hydroxytyrosol. Such encapsulated compositions include microcapsules which contain within their interior, both benzoin and olive oil extract which latter material contains hydroxytyrosol, and which may optionally contain one or more further materials.

In a still further aspect of the invention there is provided an encapsulated composition which comprises both benzoin and hydroxytyrosol, which latter material may have been separated or extracted from a naturally occurring source, i.e., olive oil and/or which may have been synthetically produced. Such encapsulated compositions include microcapsules which contain within their interior, both benzoin and hydroxytyrosol, and which may optionally contain one or more further materials.

A preferred delivery method for the treatment would be to provide both benzoin and olive oil extract which latter material contains hydroxytyrosol in microcapsules, or alternatively, to provide both benzoin and hydroxytyrosol in microcapsules, as a microencapsulated product or material, e.g, “Thin-Skin Microcapsules” (ex. Capsulent). Thin-Skin Microcapsules are nano-thin capsules that act as a tunable, targeted delivery system. The capsules are composed of natural plant-derived polysaccharides, like cellulose or alginate, and have a diameter of 0.5 to 20 microns. An SEM image of the capsules are illustrated on FIG. C.

In order to make the capsules, a primary emulsion of benzoin and olive fruit extract and/or hydroxytyrosol is first made in an aqueous solution. The polysaccharide capsule wall material is added in a second solution, which instantaneously precipitates a nano-thin, flexible wall around each droplet of primary emulsion to form the capsule using a cationic quaternary amine. Of course other encapsulation materials and techniques which are similarly effective in producing encapsulated (e.g., microencapsulated) compositions which comprise both benzoin and the olive oil extract which latter material contains hydroxytyrosol and/or hydroxytyrosol, can also be used in place of the foregoing described “Thin-Skin Microcapsules”. The capsules may then be combined with one or more further constituents or materials, e.g, in a carrier composition such as a thickened aqueous composition, or in a sprayable composition, and used for topical application. The capsules are charged in order to enhance their attraction to the skin, which is also slightly charged, in order to increase the attraction of and the retention of the capsules on a topical surface, e.g., skin.

Whereas according to the foregoing the formation of the capsules takes place in a bulk, aqueous phase and the dispersed organic phase comprises microdroplets of the benzoin and olive fruit extract and/or hydroxytyrosol which are preferably mutually miscible or soluble and thus are concurrently present within the interior of the formed capsules, preferably microcapsules, such is not a limitation of the present invention and it is to be understood that treatment compositions may comprise one or more capsules, preferably microcapsules, containing the benzoin as well as further containing one or more capsules, preferably microcapsules, containing the olive oil extract, as separate species of capsules, which are however concurrently present within a treatment composition. According to the foregoing recited process for the formation of capsules, preferably microcapsules, from an aqueous bulk phase having dispersed within the benzoin and olive fruit extract and/or hydroxytyrosol in the form of droplets, which due to good mutual miscibility are expected to mix and thus be concurrently present within the capsules at the conclusion of the process, it is also expected that a portion of the of the benzoin and olive fruit extract and/or hydroxytyrosol may not have mixed, and have separately formed one or more capsules, preferably microcapsules, containing the benzoin as well as further containing one or more capsules, preferably microcapsules, containing the olive oil extract and/or hydroxytyrosol, as separate species of capsules which are concurrently present with the majority of the formed capsules, preferably microcapsules, containing both the benzoin and olive fruit extract and/or hydroxytyrosol. However, it is expected that the total amounts of the one or more capsules, preferably microcapsules, containing the benzoin as well as further containing one or more capsules, preferably microcapsules, containing the olive oil extract, as separate species of capsules are only present as a small proportion, either as a numerical fraction or as a weight fraction of the total capsules, preferably microcapsules, formed from this process.

It is also within the scope of the present invention to form capsules, preferably microcapsules containing the benzoin, but not the olive oil extract or hydroxytyrosol, in a separate encapsulation step or process, from a further step or process used to form capsules, preferably microcapsules containing the olive oil extract and/or hydroxytyrosol, but not the benzoin. After the formation of these separately formed one or more capsules, preferably microcapsules, containing the benzoin and the separately formed one or more capsules, preferably microcapsules, containing the olive oil extract and/or hydroxytyrosol, both of these species of capsules may be separately or jointly provided to or included within a treatment composition. In such an embodiment, the benzoin and the olive oil extract and/or hydroxytyrosol may be separately released from the separate species of capsules, but these materials are mixed within the treatment composition and/or on the locus or surface upon which they are applied and as a mixture provide the advantageous benefits recited elsewhere herein.

It is also to be understood that with respect to the foregoing, that one or more further materials may be concurrently present within the capsules, preferably microcapsules, concurrently with the benzoin and with the olive oil extract, alternatively with the benzoin and hydroxytyrosol, may be produced according to any of the processes described.

The capsules, e.g., microcapsules, can be formulated to have release mechanisms such as diffusion, evaporation, temperature, permeation, targeted, pH, osmotic, biodegradation, photochemical release, or the capsules can be ruptured with force. In the case of this treatment, when the capsules come into contact with the skin, the skin's natural lipids and enzymes slowly degrade their plant-derived polysaccharide wall. The capsules can thus be formulated to provide controlled delivery of benzoin and olive fruit extract and/or hydroxytyrosol over time. The reservoir action of these capsules can significantly extend the effect of the active that is encapsulated, as seen in the graph according to FIG. A. These capsules can also be constructed down to a nano-scale to penetrate deep into the skin. Thin-Skin Microcapsules are polydisperse, and are advantageously used as they may be produced in a range of sizes. Capsules, e.g. microcapsules, may be selectively formed with regard to their size in order to achieve a desired surface packing effect. Desirably such surface packing effects are preferred as maximizing the surface packing effects, to be great than monolayer or monodisperse delivery levels, increase the surface density of the applied capsules. A graphical representation of this phenomenon is illustrated on FIG. B. The capsules' charged, polydisperse aspect, and controlled release abilities lead to better utilization of the benzoin and olive fruit extract that is encapsulated. Thin-Skin Microcapsules provide the advantage of having capsule walls which may be very small, e.g., of nanometer thickness, thus allowing fragrance or active diffusion after product dries. As explained previously, the capsule wall is produced in a single step around the dispersed suspended droplets comprising the benzoin and olive fruit and does not use toxic cross-linkers that must be rinsed away.

Capsules, or microcapsules which comprise the benzoin and olive oil extract and/or comprising benzoin and hydroxytyrosol may be used as constituents in any of the forms and product formats, especially in any of the personal care compositions, cosmetic compositions or wound dressings as described above.

Treatment compositions and topical treatment compositions which comprise both benzoin and an olive oil extract having a high proportion of hydroxytyrosol, and or hydroxytyrosol may be advantageously used to treat one or more of one or more of the following physical conditions in a mammal (e.g., human), especially when topically applied in effective amounts: To treat skin abrasions, cuts, wounds as well as skin inflammation
(ii) To soothe dryness and moisturize skin
(iii) To act as an anti-acne treatment
(iv) To act as an antibacterial treatment
(v) To treat the appearance of aged, wrinkled, rough, flaky, or photo-damaged skin
(vi) To decrease skin fragility
(vii) To restore skin luster, tone, and texture
(viii) To improve skin firmness/plumpness
(ix) To prevent or reverse loss of collagen
(x) To prevent skin atrophy
(xi) To minimize pores
(xii) To soothe and treat sunburned skin
(xiii) To act as an antioxidant treatment for skin
(xiv) To even skin color

Treatment compositions and topical treatment compositions which comprise: microencapsulated benzoin and olive fruit extract, especially preferably wherein the olive fruit extract comprises hydroxytyrosol and/or hydroxytyrosol and further preferably wherein the foregoing constituents are microencapsulated, preferably within Thin-Skin Microcapsules, may be advantageously used in the to treat one or more of the following physical conditions in a mammal (e.g., human), especially when topically applied in effective amounts:
(i) To treat skin abrasions, cuts, wounds as well as skin inflammation
(ii) To soothe dryness and moisturize skin
(iii) To act as an anti-acne treatment
(iv) To act as an antibacterial treatment
(v) To treat the appearance of aged, wrinkled, rough, flaky, or photo-damaged skin
(vi) To decrease skin fragility
(vii) To restore skin luster, tone, and texture
(viii) To improve skin firmness/plumpness
(ix) To prevent or reverse loss of collagen
(x) To prevent skin atrophy
(xi) To minimize pores
(xii) To soothe and treat sunburned skin
(xiii) To act as an antioxidant treatment for skin
(xiv) To even skin color

The treatment system which comprises: microencapsulated benzoin and an olive fruit extract which comprises hydroxytyrosol, and/or hydroxytyrosol, and further preferably wherein the foregoing constituents are microencapsulated, preferably within Thin-Skin Microcapsules, may be advantageously incorporated as an additive or constituent, or otherwise form a part of any of a number of treatment compositions, e.g, topical treatment compositions, which are useful the treatment of one or more of the following physical conditions in a mammal (e.g., human):
(i) To treat skin abrasions, cuts, wounds as well as skin inflammation
(ii) To soothe dryness and moisturize skin
(iii) To act as an anti-acne treatment
(iv) To act as an antibacterial treatment
(v) To treat the appearance of aged, wrinkled, rough, flaky, or photo-damaged skin
(vi) To decrease skin fragility
(vii) To restore skin luster, tone, and texture
(viii) To improve skin firmness/plumpness
(ix) To prevent or reverse loss of collagen
(x) To prevent skin atrophy
(xi) To minimize pores
(xii) To soothe and treat sunburned skin
(xiii) To act as an antioxidant treatment for skin
(xiv) To even skin color

### EXAMPLES

Compositions comprising both benzoin and olive fruit extract which extract comprises hydroxytyrosol were produced, and evaluated as generally set forth below.

Benzoin (CAS #119-53-9), laboratory grade preparation obtained from a suitable supplier (e.g., Sigma Aldrich Co.), was utilized.

An olive oil extract, certified as containing 25% hydroxytyrosol (CAS #10597-60-1), obtained from a suitable supplier, was used in the following.

A human keratinocyte cell line, HaCaT, derived from the epidermis of skin, was cultured in this experiment to test for the presence of MMP-9 and fibronectin. A dermal fibroblast primary cell culture, derived from the dermis, was cultured in this experiment to test for the presence of collagen and elastin.

Cell Culture:

Human keratinocytes (HaCaT) and dermal fibroblasts were grown in Dulbecco's Modified Eagle's Medium (DMEM) media with 10% fetal bovine serum (Gibco's) and were incubated at 37° C. in 5% CO₂. The cells were trypsinized and their media were changed 2 times per week.

Cells were distributed into tissue culture plates at approximately 1×10⁵ cells/well.

Chemical and Experimental Design:

The benzoin obtained from Sigma Aldrich was diluted using 99.85% dimethyl sulfoxide into the following solutions: 0%, 0.5 μM, 0.05 μM. The olive fruit extract was diluted in distilled water into the following solutions: 0%, 0.001%, 0.002%.

Cell Viability Assay:

An MTS assay was used to measure the viabilities of human keratinocytes and dermal fibroblasts under the treatment. Cells were plated at approximately 1×10⁵ cells/well. After 24 hours, the cells were exposed to different concentrations of benzoin and olive fruit extract in replicates of five, and incubated for an additional 24 hours. Cell viability was then determined using the CellTiter 96® AQᵤₑₒᵤₛ One Solution Cell Proliferation Assay (Promega, Madison, Wis., USA). This assay is based on the conversion of tetrazolium to form Formosan, a colored product, by reducing NADPH. The assay was performed by adding 15 of the CellTiter 96® AQᵤₑₒᵤₛ One Solution Reagent to 100 μL of culture media and incubating the plate for 2 hours. To measure the conversion of tertrazolium to formazan product, the absorbance was recorded at 490 nm using a 96-well microplate reader (BioTek Elx808; BioTek, Winno ski, Vermont, USA).

DPPH Assay:

The free radical scavenging activity of olive fruit extract was measured using 2,2-diphenyl-1-picrylhydrazyl (DPPH), which is lavender-colored and composed of free radical molecules. The ability of olive fruit extract to reduce the free radical would be shown by a decrease in the lavender solution color. 50 μL of varying concentrations of olive fruit extract were combined with 50 μL of a 1 mM DPPH solution. A spectrophotometer was used to assay these results at 515 nm after two hours of incubation. The reduction of DPPH is shown below:

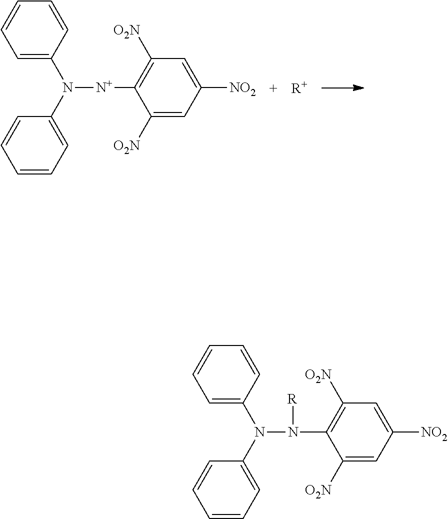

MMP-9 Assay:

The presence of MMP-9 was measured using an enzyme-linked immunsorbent assay (RayBio, Norcross, Ga., USA). Cells were plated at approximately 1×10⁵ cells/well and were treated with varying concentrations of Benzoin and olive fruit extract in replicates of three. The assay was carried out according to manufacturer's protocol. Absorbance was determined at 490 nm.

Fibronectin Assay:

The presence of fibronectin was measured using an enzyme-linked immunsorbent assay (Millipore, Billerica, Mass., USA). Cells were plated at approximately 1×10⁵ cells/well and were treated with varying concentrations of Benzoin and olive fruit extract in replicates of three. The assay was carried out according to manufacturer's protocol. Absorbance was determined at 450 nm.

Collagen Assay:

The presence of collagen was measured using an enzyme-linked immunsorbent assay (RayBio, Norcross, Ga., USA). Cells were plated at approximately 1×10⁵ cells/well and were treated with varying concentrations of Benzoin and olive fruit extract in replicates of three. The assay was carried out according to manufacturer's protocol. Absorbance was determined at 490 nm.

Elastin Assay:

The presence of elastin was measured using an enzyme-linked immunsorbent assay (RayBio, Norcross, Ga., USA). Cells were plated at approximately 1×10⁵ cells/well and were treated with varying concentrations of Benzoin and olive fruit extract in replicates of three. The assay was carried out according to manufacturer's protocol. Absorbance was determined at 490 nm.

Data Analysis:

Data were analyzed using Excel (Microsoft, Redmond, Wash., USA). All assays were repeated at least twice, with sample size for each condition at least n=3. The Student's t-test was used to determine significance, with a value of p<0.05 considered to be statistically significant.

The results of the foregoing analysis are disclosed on FIG. 1 which discloses and depicts the effect of benzoin and olive fruit extract at various concentrations on the viability of human keratinocytes and dermal fibroblasts. As can be seen from the FIG. 1, benzoin and olive fruit extract, when combined, caused statistically significant proliferation in human keratinocytes. All concentrations of benzoin and olive fruit extract were nontoxic to both cell lines. Data for each cell line was normalized to the untreated control (0 μM benzoin and 0% olive fruit extract) for that cell line. Bars on FIG. 1 are means+/−SD (n=5). * denotes p<0.05 when compared to control.

On FIG. 2 is disclosed and depicted the effect of benzoin and the aforesaid olive fruit extract at various concentrations on the presence of MMP-9 in human keratinocytes. As is evident from the foregoing FIG. 2, in this experiment performed solely in human keratinocytes.

MMP-9 is most active in the epidermis, where HaCaT cells originate. The combined benzoin and olive fruit extract compositions statistically significantly decreased levels of MMP-9. Data was normalized to the results of the control (0 μM benzoin and 0% olive fruit extract). Bars on FIG. 2 are means+/−SD (n=3). * denotes p<0.05 when compared to control.

FIG. 3 discloses and depicts the effect of benzoin and the olive fruit extract containing a high proportion of hydroxytyrosol at various concentrations on the presence of collagen in dermal fibroblasts. The experiment was performed solely on dermal fibroblasts since collagen is more prevalent in the dermis, where dermal fibroblasts originate. As is understood from a consideration of FIG. 3, compositions comprising both benzoin and olive fruit extract, demonstrate significantly increased collagen in dermal fibroblasts. Data was normalized to the results of the control (0 μM benzoin and 0% olive fruit extract). Bars are means+/−SD (n=3). * denotes p<0.05 when compared to control.

FIG. 4 discloses and depicts the effect of benzoin and the olive fruit extract on the presence of elastin in dermal fibroblasts. This experiment was performed solely on dermal fibroblasts since elastin is most prevalent in the dermis, where dermal fibroblasts originate. As evident from FIG. 4, compositions comprising benzoin and the aforesaid olive fruit extract, in three of four combinations, significantly increased the levels of elastin. Data was normalized to the results of the control (0 μM benzoin and 0% olive fruit extract). Bars of FIG. 4 are means+/−SD (n=3). * denotes p<0.05 when compared to control.

FIG. 5 discloses and depicts the effect of benzoin and the aforesaid olive fruit extract at various concentrations on the presence of fibronectin in human keratinocytes. This experiment was performed solely on human keratinocytes since fibronectin is most active in the epidermis, where human keratinocytes originate. As seen from the foregoing FIG. 5, the combination of benzoin and olive fruit extract having a significant proportion of hydroxytyrosol had significantly increased the levels of fibronectin in human keratinocytes at the highest concentrations. Data was normalized to the results of the control (0 μM benzoin and 0% olive fruit extract). Bars of FIG. 5 are means+/−SD (n=3). * denotes p<0.05 when compared to control.

A scratch assay was also performed to qualitatively observe the wound healing abilities of the treatment. As seen from on FIGS. 6 and 7, after scratching the plated dermal fibroblasts to simulate a wound, the proliferation and migration of the cells were observed using phase contrast microscopy. The assay demonstrated that benzoin and the aforesaid olive fruit extract caused faster recovery (coverage of cells across the scratched region) compared to the control.

Benzoin and olive fruit extract, which olive fruit extract comprised a high proportion of hydroxytyrosol, both alone and combined, were nontoxic to human keratinocytes and dermal fibroblasts. Furthermore, combinations of benzoin and olive fruit extract induced significant proliferation in human keratinocytes. These results support benzoin and olive fruit extract's wound healing abilities, since cell growth and proliferation are vital to skin repair.

The levels of MMP-9 significantly decreased in the presence of the combination of benzoin and the olive fruit extract which contained a high proportion of hydroxytyrosol. A decrease in MMP-9 will lead to a decrease in degradation done by MMPs to the extracellular matrix. It is interesting to note that the decrease of MMP-9 was less than additive when benzoin and olive fruit extract were combined. This shows that a higher—and more toxic—concentration of one component alone would have to be used to yield the same effect as a combination of the components at lower, less toxic concentrations. Such also appears to indicate that a synergistic improvement is attained with the combination of benzlin and the olive fruit extract. Benzoin and the olive fruit extract, when combined, also significantly increased the levels of collagen and elastin in dermal fibroblasts. These two proteins give firmness and elasticity to the skin, but are broken down by MMP-9. Therefore, these results show that the unique combination of benzoin and the olive fruit extract increases the presence of collagen and elastin via the downregulation of MMP-9. A decrease in MMP-9 is also often associated with a decrease in oxidative stress, since the former induces the latter. Therefore, these results demonstrate that the combination of benzoin and the aforesaid olive fruit extract acts as a free radical scavenger, and decreases oxidative stress via the downregulation of MMP-9. This shows that the aforementioned combination holds great potential for a wound healing treatment, since a decrease in oxidative stress is crucial to skin repair.

The combination of benzoin and olive fruit extract having a high amount of hydroxytyrosol also clearly demonstrated faster and more efficient coverage of a wound, by inducing the proliferation and migration of plated cells across a scratched region. This rapid proliferation and migration is significant for wound repair, and shows the capability of the benzoin and olive fruit extract combination as a repair treatment.

The presence of benzoin and the aforesaid olive fruit extract showed a trend towards a modest increase of fibronectin in human keratinocytes. A significant increase was observed in the combination of the highest concentrations of benzoin and the olive fruit extract. A result showing a modest increase in fibronectin is preferred, thus minimizing the concern of possible scar tissue formation.

This study consistently showed that the combination of benzoin and the olive fruit extract curtails many of the destructive reactions that occur in the presence of a skin wound. The combination of benzoin and olive fruit extract induced a significant decrease in MMP-9, which led to a significant increase in the proteins collagen and elastin. A decrease in MMP-9 will cause less degradation to the extracellular matrix while an increase in collagen and elastin will give strength and elasticity to skin. These results show that the combination of benzoin and theolive fruit extract having a high proportion of hydroxytyrosol are halting the body's destructive outburst to a wound so that the skin can repair itself naturally. The unique combination of benzoin and olive fruit extract which contained hydroxytyrosol in a significant proportion provides a skin repair treatment due to its wound healing effects on the skin.

The foregoing results demonstrate the surprising benefits provided by compositions containing both benzoin and hydroxytyrosol even when the amount of hydroxytyrol was in a substantially minor amount as compared to the amount of benzoin present, e.g., where the relative ratios/proportions of benzoin:hydroxytyrosol (preferably wt/wt) was in the range of 10,000-500:1, more preferably 5000-500:1, yet more preferably 35000-1000:1 A particularly preferred relative ratio of benzoin:hydroxytyrosol (wt/wt), namely 1620:1, is disclosed with reference to the examples.

### Example—Shampoo Composition

A shampoo composition reported on Table F1 was formed from the indicated constituents which were present in the indicated amounts. The constituents were used “as supplied” and where not provided as a “100% wt. actives” constituent, the amount of “% wt. actives” are indicated in parenthesis.

TABLE F1

| Constituent | CAS # | Description | % wt. |
|---|---|---|---|
| Water | 7732-18-5 | | 59.1 |
| Sodium Lauryl Sulfate (30% | 151-21-3 | Anionic | 10.0 |
| active) | | Surfactant | |
| Sodium Laureth-2 Sulfate | 9004-82-4 | Anionic | 26.0 |
| (27% active) | | Surfactant | |
| Cocamide DEA | 68603-42-9 | Non-Ionic | 2.0 |
| | | Surfactant | |
| Skin Repair Complex | Blend | Active Complex | 1.0 |
| Glycol Distearate | 627-83-8 | Glycol Ester | 1.0 |
| Preservative | | | 0.4 |
| Fragrance | | Fragrance | 0.5 |
| Sodium Chloride | 7647-14-5 | Salt | q.s. |
| Citric acid (if needed) | 77-92-9 | Acid | q.s. |

The shampoo composition was formed by first heating the water to approx. 50° C. in a suitable laboratory beaker. Next under stirring conditions was added the cocoamide DEA, then the glycol distearate. Heating was ceased and the as the composition was cooling to room temperature, was then added the “Skin Repair Complex”, the anionic surfactants and then the remaining constituents with the fragrance composition being added last. Stirring continued until the composition was homogeneous, after which it was ready for use. The composition was viscous and had a viscosity of at least about 70 cPs, and was pourable.

The “Skin Repair Complex” material used in the present composition (F1) as well as in other compositions (F2, F3 and F5) was a proprietary blend of synthetic benzoin with an olive oil extract which contained 25% hydroxytyrosol. It was first formed by mixing the olive oil extract containing as well as other components of olive oil and inert solvents, to which was added the synthetic benzoin under continuous mixing which continued until a clear and homogenous composition resulted. The Skin Repair Complex thus formed comprised 0.00025% wt. hydroxytyrosol and 0.405% wt. benzoin with the remaining balance to 100% wt. being other components of olive oil, and inert solvents.

### Example—Hair Conditioner

A hair conditioner composition reported on Table F2 was formed from the indicated constituents which were present in the indicated amounts. The constituents were used “as supplied” and where not provided as a “100% wt. actives” constituent, the amount of “% wt. actives” are indicated in parenthesis.

TABLE F2

| Constituent | CAS # | Description | % wt. |
|---|---|---|---|
| Cetyl Alcohol | 36653-82-4 | Stearyl Alcohol | 3.0 |
| Quaternium-80 | 134737-05-6 | Siloxane/Silicone | 1.0 |
| Steareth-21 | 7651-02-7 | Cationic Surfactant | 1.0 |
| Ethylhexyl Palmitate | 14858-73-3 | Ester | 1.0 |
| Skin Repair Complex | Blend | Active Complex | 1.0 |
| Ceteareth-25 | 68439-49-6 | Ethoxylated Alcohol | 0.5 |
| Water | 7732-18-5 | | 88.0 |
| Propylene Glycol | 57-55-6 | Glycol | 2.0 |
| Citric Acid | 77-92-9 | Acid | 0.3 |
| Hydrolyzed Soy Protein | 68607-88-5 | Protein | 1.5 |
| Preservative | | | 0.7 |

The hair conditioner composition was formed by first forming a “Phase A” premixture which was formed by adding the cetyl alcohol, Quaternium-80, steareth-21, ethylhexyl palmitate and the Skin Repair Complex in a suitable laboratory beaker and under stirring, heating these materials to 75° C. to form a homogenous composition. A “Phase B” premixture was formed by adding the ceteareth-25, propylene glycol, citric acid and water to another suitable laboratory beaker and under stirring, heating these materials to 75° C. to form a homogenous composition. A “Phase C” premixture was formed by combining under stirring conditions the hydrolyzed soy protein and the preservative in another suitable laboratory beaker to form a homogenous composition. Thereafter the Phase A premixture was combined with the Phase B premixture under stirring conditions, which was allowed to cool to approximately 65° C. under moderate stirring conditions which stirring continued until the combined premixtures formed a homogenous composition. Cooling was allowed to continued, and when the combined premixtures were approximately 40° C., was then added a the Phase C premixture under stirring, which continued until the final hair conditioner composition cooled to room temperature, (approximately 20-22° C.)

### Example—Body Cream Formulation

A body cream composition reported on Table F3 was formed from the indicated constituents which were present in the indicated amounts. The constituents were used “as supplied” and where not provided as a “100% wt. actives” constituent, the amount of “% wt. actives” are indicated in parenthesis.

TABLE F3

| Raw Material | CAS # | Description | % wt. |
|---|---|---|---|
| Water | 7732-18-5 | | 47.00 |
| Dehydroxyxanthan Gum | 11138-66-2 | Gum | 0.50 |
| Hydrogentated Lecithin | 92128-87-5 | Lecithin | 4.00 |
| Sodium Benzoate | 532-32-1 | Benzoate | 0.35 |
| Potassium Sorbate | 24634-61-5/590-00-1 | | 0.15 |
| Propanediol | 504-63-2/26264-14-2 | Diol | 4.00 |
| Helianthus Annuus | 8001-21-6 | Seed Oil | 17.0 |
| Seed Oil | | | |
| Hydrogenated Coconut Oil | 84836-98-6 | Oil | 2.0 |
| Carnauba Wax | | Wax | 2.0 |
| Skin Repair Complex | Proprietary Blend | Active | 1.0 |
| | | Complex | |
| Naviance Tapioca LM | 9005-25-8 | | 22.0 |
| Cook | | | |

The body cream formulation was formed by first forming a “Phase A” premixture containing the water, dehydroxyxanthan gum, hydrogenated lecithin, sodium benzoate, potassium sorbate and propanediol. To a suitable laboratory beaker was added water which was heated to 80° C. Thereafter under stirring conditions was slowly added the dehydroxyxanthan gum until it was fully dispersed. Thereafter under continued stirring were added the remaining constituents of Phase A, and mixing continued to homogeneity. A “Phase B” premixture containing the heliantus annus seed oil, hydrogenated coconut oil, carnauba was and the skin repair complex was formed by combining these constituents under stirring in a further laboratory beaker and heating the premixture to 80° C. Thereafter the Phase B premixture was added to the Phase A premixture under stirring which continued for 15 minutes while the temperature was maintained at 80° C. Subsequently the Naviance Tapioca LM Cook was added to this combined Phase A and Phase B premixture under stirring, and while stirring the final body cream composition was allowed to cool to room temperature.

### Example—Body Wash (II)

A body wash composition reported on Table F4 was formed from the indicated constituents which were present in the indicated amounts. The constituents were used “as supplied” from their respective supplier; the tradenames are provided to aid in the identification of the specific constituent used.

TABLE F4

| Constituent (tradename) | Constituent (description) | % wt. |
|---|---|---|
| DI Water | Deionized Water | 60.95 |
| Sulfochem ES-2PK | Sodium Laureth Sulfate | 22.00 |
| Carbopol Aqua SF-1 Polymer | Acrylates Copolymer | 7.00 |
| Chembetaine CAD | Cocamidopropyl Betaine | 7.00 |
| Glucamate VLT | PEG-120 Methyl Glucose | 1.00 |
| | Trioleate & Propanediol | |
| Sodium Hydroxide (20% soln) | Sodium Hydroxide | 1.00 |
| Skin Repair Complex - | Proprietary Blend | q.s. |
| Encapsulated* | | |
| Neolone 950 | Methylisothiazolinone | 0.05 |
| | TOTAL = | 100.00 |

*sufficient (approx. 2.5% wt.) “Skin Repair Complex - Encapsulated” was added to provide an effective concentration of 1% wt. of the encapsulated “Skin Repair Complex” material, such that the Table F4 composition contained 0.00025% wt. hydroxytyrosol and 0.405% wt. benzoin

In the composition of Table F4 (as well as in the following composition of Table F5) the “Skin Repair Complex—Encapsulated” was an encapsulated blend of synthetic benzoin with olive oil extract which contained hydroxytyrosol, which encapsulated material was the same as the “Skin Repair Complex” used in the formulations of Tables F1, F2, F3 and F5. The encapsulated material (also referred to as “Skin Repair Complex”) was formed as previously discussed with reference to the formulation of Table F1. It was first formed by mixing the olive oil extract containing as well as other components of olive oil and inert solvents, to which was added the synthetic benzoin under continuous mixing which continued until a clear and homogenous composition resulted. The Skin Repair Complex thus formed comprised 0.00025% wt. hydroxytyrosol and 0.405% wt. benzoin with the remaining balance to 100% wt. being other components of olive oil, and inert solvents. Thereafter an aliquot of the Skin Repair Complex was microencapsulated by dispersion of 10% wt. of the oil composition in 0.1% sodium alginate containing 0.5% sorbitan monooleate and 0.5% polysorbate 20, using a magnetic stirrer, and subsequent precipitation of an encapsulating wall by addition of an equal volume of 0.05% benzalkonium chloride, as taught in U.S. Pat. No. 8,039,015 to Speaker the contents of which are herein incorporated by reference. The resultant “Skin Repair Complex”—Encapsulated had a average particle diameter of 1-50 microns, with a nano-thin wall.

The body wash composition was formed by combining measured amount of the indicated constituents, except for the preservative, into a clean laboratory beaker at room temperature under gentle stirring conditions. This proceeded until the mixture was homogenous, after which the preservative was added and stirring continued until homogenous, after which the body wash was ready for use.

### Example—Lotion

A topical lotion composition reported on Table F5 was formed from the indicated constituents which were present in the indicated amounts. The constituents were used “as supplied” from their respective supplier; the tradenames are provided to aid in the identification of the specific constituent used.

TABLE F5

| Constituent (tradename) | Constituent (description) | % wt. |
|---|---|---|
| DI Water | Deionized Water | 85.95% |
| Carbopol Ultrez 10 Polymer | Carrageenan | 1.00% |
| Ritabate 20 | Polysorbate 20 | 0.50% |
| Jeechem SMO | Sorbitan Monooleate | 0.50% |
| CRODAMOL ™ GTCC | Caprylic/Capric Triglyceride | 10.00% |
| Sodium Hydroxide (20% | Sodium Hydroxide | 1.00% |
| soln) | | |
| Skin Repair Complex - | Proprietary Blend | 1.00% |
| Encapsulated | | |
| Neolone 950 | Methylisothiazolinone | 0.05% |

In the composition of Table F5 (as well as in the following composition of Table F4) was used the “Skin Repair Complex—Encapsulated” as described previously with reference to the formulation of Table F4. The topical lotion composition was formed by first forming a first “Phase A” premixture, by first combining the water, and the carageenan gum with the surfactants in a laboratory beaker under stirring conditions at room temperature and stirring continued until the mixture was homogenous. A separate “Phase B” premixture was formed by combining the remaining constituents, except for the preservative, in a laboratory beaker under stirring conditions at room temperature and stirring continued until the mixture was homogenous. Thereafter the Phase A premixture was combined with the Phase B premixture under stirring conditions at room temperature until the resultant mixture was homogenous, and then the preservative was added and again, stirring continued until the final topical lotion composition was homogenous, after which it was ready for use.

1. (canceled)

2. (canceled)

3. (canceled)

4. (canceled)

5. (canceled)

6. (canceled)

7. (canceled)

8. (canceled)

9. (canceled)

10. (canceled)

11. A treatment composition which initiates the generation of human keratinocytes on or within the treated mammalian skin in the locus of the skin wherein the treatment composition has been applied, which treatment composition comprises: microencapsulated benzoin and microencapsulated hydroxytyrosol and/or an olive oil extract which contains hydroxytyrosol and wherein the capsules comprise cellulose.

12. A treatment composition according to claim 10, wherein the treatment composition is a topical treatment composition effective in inducing the generation of human keratinocytes on the treated in the locus of the skin wherein the treatment composition has been applied.

13. A treatment composition according to claim 10, wherein the treatment composition is a skin repair composition.

14. A treatment composition according to claim 10, wherein the treatment composition is a wound treatment composition.

15. A treatment composition according to claim 10, wherein the benzoin and the hydroxytyrosol are present in a respective weight ratio range of 35000-500:1.

16. A treatment composition according to claim 15, wherein the benzoin and the hydroxytyrosol are present in a respective weight ratio range of 10000-500:1.

17. A treatment composition according to claim 16, wherein the benzoin and the hydroxytyrosol are present in a respective weight ratio range of 5000-1000:1.

18. A method for the treatment of mammalian skin, which method comprises the step of:
applying a composition according to claim 10 to mammalian skin, and inducing, increasing or initiating the generation of human keratinocytes on or within the treated skin in the locus of the skin wherein the treatment composition has been applied.

19. A treatment composition according to claim 10, wherein the microencapsulated benzoin and a microencapsulated olive oil extract are both present within a single, charged capsule.

20. A treatment composition according to claim 10, wherein the microcapsules have a diameter of 0.5 to 20 microns.

21. A treatment composition according to claim 10, wherein the treatment composition is selected from the group consisting of: liquids, thickened liquids, gels, pastes, salves, cremes, tablets, bars, foaming sprayable compositions and non-foaming sprayable compositions, pressurized compositions and non-pressurized compositions.

22. A treatment composition according to claim 10, wherein the treatment composition is selected from the group consisting of; shampoo, conditioner, shower gel, hair spray, hair pomade, hair gel, bodywashes, liquid soaps, and personal care products.

23. A treatment composition according to claim 10 which is a cosmetic composition.

24. A treatment composition according to claim 10 which s adapted to be applied to the face, hands, or skin elsewhere on the human body.

25. A treatment composition according to claim 10 wherein the capsules are charged capsules.
